(19) United States
(12) Patent Application Publication
(10) Pub. No.: US 2022/0266223 A1
(43) Pub. Date: Aug. 25, 2022

(54) BISMUTH MOLYBDATE-BASED CATALYST, PROCESS FOR THE PRODUCTION THEREOF AND USE OF THIS CATALYST IN THE OXIDATION OF PROPENE TO ACROLEIN

(71) Applicants: ADISSEO FRANCE S.A.S., ANTONY (FR); CENTRE NATIONAL DE LA RECHERCHE SCIENTIFIQUE, PARIS (FR); UNIVERSITE CLAUDE BERNARD LYON 1, VILLEURBANNE (FR)

(72) Inventors: Virginie BELLIERE-BACA, MILLERY (FR); Jean-Marc MILLET, LYON (FR); Aziz CHERIFI, Genay (FR); Catarina ROCHA, Communay (FR)

(21) Appl. No.: 17/631,717

(22) PCT Filed: Jul. 31, 2020
(86) PCT No.: PCT/FR2020/051408

§ 371 Date: Jan. 31, 2022

(30) Foreign Application Priority Data
Jul. 31, 2019 (EP) 19189424.5
Oct. 23, 2019 (FR) 19/11836

(51) Int. Cl. B01J 23/28; B01J 37/04; B01J 37/34; B01J 23/881; C07C 45/28

(52) U.S. Cl. CPC B01J 23/28; B01J 37/04; B01J 37/346; B01J 23/881; C07C 45/28; C07C 2523/881; C07C 2523/28

(57) ABSTRACT

A method for producing a multiphase mixed-oxide catalyst including at least one active phase based on bismuth molybdate and one co-catalyst based on iron molybdate and at least one amongst the two elements cobalt and nickel, includes the following steps:

preparing a mixture of the precursors of said-mixed oxides in a solvent,

making said precursors react through a microwave-assisted hydrothermal reaction, and

isolating the mixed oxides to obtain the catalyst.

A catalyst and a catalytic system prepared in this manner are related to the method as well as the uses of this catalyst and of this catalytic system, in particular in the oxidation of propene into acrolein.

### TECHNICAL FIELD

The present disclosure concerns a method for preparing a multiphase mixed-oxide catalyst based on bismuth molybdate, a catalyst and a catalytic system obtained in this manner and the uses of these in different oxidation reactions.

### BACKGROUND

The performances of the catalyst or of the catalytic system according to the disclosure are set out hereinafter in the controlled oxidation reaction of propene into acrolein. These are not restricted thereto and are interesting in particular in the oxidative dehydrogenation of butene into butadiene, the oxidation of isobutylene into methacrolein, the ammoxidation of propene into acrylonitrile and the ammoxidation of isobutylene into methacrylonitrile. All these well-known reactions are implemented on an industrial scale as they supply source monomers for the production of many polymers and precursors that are essential in organic synthesis. For this reason, they are continuously the object of researches aiming at improving their performances and reducing their ecological impact.

The controlled oxidation of propene that is represented hereinbelow leads to acrolein which is an intermediate for the synthesis of methionine and derivatives thereof, widely used in animal nutrition.

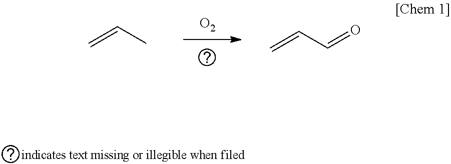

It is performed in the presence of a multiphase mixed-oxide catalyst, composed by at least four metallic elements: molybdenum and bismuth, which form the selective phases of bismuth molybdate, and a combination of metals in the oxidation state +2 (in general Ni or Co) and the oxidation state +3 (in general iron) and molybdenum which form phases that strongly enhance the catalytic activity by promoting the re-oxidation of the catalyst. Thus, it meets the minimum formula Mo(Co/Ni)FeBiO which is completed by various elements present as doping elements and/or in the form of oxides or molybdate, in order to improve the properties of the catalyst, such as selectivity, thermal stability, mechanical stability . . . .

The document US2019/076829A1 describes such a catalyst and, inter alia, a catalyst meeting the following formula:

Mo₁₂Bi₁₋₄Co₄₋₁₀Fe₁₋₄Ni₀₋₄K₀₋₂Oₓ

as well as a method for preparing it comprising the following steps:

an aqueous mixture of the precursors of the elements of the catalyst is prepared in proper contents so as to reach the stoichiometry,

after setting of the pH of the mixture to 5.5-8.5, said precursors are reacted through a hydrothermal reaction in an autoclave at a temperature from 100° C. to 600° C., for 5.5 hours to 48.5 hours, and then

the catalyst is retrieved.

### SUMMARY

Such a preparation requiring long reaction times, the authors have developed a method for producing a catalyst of the aforementioned type, by replacing the hydrothermal reaction hereinabove with a microwave-assisted hydrothermal reaction. They have discovered that, while allowing reducing the reaction duration considerably, the obtained catalyst has unexpected characteristics, conferring better properties thereon, in particular a higher reactivity.

Thus, the disclosure concerns a method for producing a multiphase mixed-oxide catalyst comprising at least one active phase based on bismuth molybdate and one co-catalyst based on iron molybdate and at least one amongst the two elements cobalt and nickel, said method comprising the following steps:

preparing a mixture of the precursors of said mixed oxides in a solvent,

making said precursors react through a microwave-assisted hydrothermal reaction, and

isolating the mixed oxides to obtain the catalyst.

By microwaves according to the disclosure, it should be understood intermediate radiations between infrared and radio waves, namely waves whose frequency is comprised between 800 and 3000 MHz. In practice, since all wavelengths are not authorized, preference will be given, yet without the disclosure being restricted thereto, to domestic microwaves used in industrial applications with a vacuum wavelength of about 12 cm, namely a frequency of about 2450 MHz, as well as microwaves used essentially in industrial applications, with a vacuum wavelength of about 33 cm, namely a frequency of about 915 MHz.

### DETAILED DESCRIPTION

The method is described hereinafter in more details, the following features may be considered separately or in any combination.

According to a particular implementation of the method of the disclosure, said precursors are reacted through a microwave-assisted hydrothermal reaction in two steps, namely: a first microwave-assisted hydrothermal reaction and a second microwave-assisted hydrothermal reaction, between which the pH of the reaction mixture obtained from the first microwave-assisted hydrothermal reaction is set to 8-8.5.

More specifically, in a first synthesis step, said precursors are added and reacted through a first microwave-assisted hydrothermal reaction, and in a second synthesis step, the pH of the reaction medium is set preferably to a value of 8-8.5, and a second microwave-assisted hydrothermal reaction is performed, and then the microwaves catalyst is isolated.

The microwave-assisted hydrothermal reaction(s) is/are preferably performed at a temperature which does not exceed 300° C., advantageously from 150° C. to 240° C.

As said before, the reaction time is significantly shortened and if the microwave-assisted hydrothermal reaction(s) could be carried out over a time period from 2 minutes to 10 hours, within a few hours, and even only a few minutes, the reaction may be almost complete.

Optionally, the catalyst prepared according to the method of the disclosure has the following features, which may be considered separately or in any combination:

it further comprises molybdenum oxide;

it is supported; in the method of the disclosure, the addition of one or several support material(s), such as silica, alumina, their mixtures, is advantageously performed before the microwave-assisted hydrothermal reaction(s); of course, an unsupported catalyst obtained according to the method of the disclosure may be supported afterwards according to any technique well known to those skilled in the art;

the active phase of the catalyst meets the following stoichiometry BiₓMo_{y}O_{z} where 2>x/y>0.5 and z is comprised between 6 and 12; still preferably this stoichiometry is selected amongst Bi₂Mo₃O₁₂, Bi₂Mo₂O₉ and Bi₂MoO₅;

the active phase of the catalyst is activated by at least one alkali metal, preferably at least potassium; in an advantageous production way, only the active phase of the catalyst is activated by one or several alkali metal(s), such as potassium;

the co-catalyst meets the following stoichiometry FeₓCo₁₋ₓMoO₄ where x is a decimal number such that 0<x<1, preferably such that 0.5≤x≤0.9;

the catalyst meets the formula MoₘCoₙNiₚFe_{q}BiᵣMₛ where M is an alkali metal, m, n, p, q, r and s are natural or decimal numbers, with m varying from 1 to 5, n and p varying independently of one another from 0 to 1 with n+p different from 0, 0<q≤1, 0<r≤3 and 0<q≤0.2.

The disclosure also concerns the catalyst obtained in this manner.

In a particular implementation of the method of the disclosure:

a mixture of the precursors of the active phase in a solvent, on the one hand, and a mixture of the precursors of the co-catalyst in the same solvent or in another solvent, on the other hand, are prepared;

the precursors of the active phase and the precursors of the co-catalyst are reacted separately, through a microwave-assisted hydrothermal reaction;

the active phase and the co-catalyst, respectively, are isolated; and

the active phase and the co-catalyst are assembled to obtain the catalyst.

Preferably, the mixture(s) of the precursors of the mixed oxides is/are obtained in one or more of the solvents selected from water, organic solvents and any combination of said organic solvents together or with water.

According to this implementation, the catalyst may be supported, said method then comprising the addition of one or several support material(s), and/or the catalyst may comprise molybdenum oxide, said method also comprising the addition of molybdenum oxide, the addition of said support material(s) and/or of the molybdenum oxide being carried out during the synthesis or the assembly of the active phase and of the co-catalyst to obtain the catalyst. The molybdenum oxide allows, on the one hand, compensating for the loss of molybdenum over time which vaporizes thereby keeping the active phase and the co-catalyst in an optimum state and, on the other hand, wetting the active phase and suppressing non-selective sites. Of course, any other phase allowing improving the properties of the catalyst may be added.

Each of the active phase and the co-catalyst, which may comprise any other phase mentioned before, is generally obtained in the form of a powder. The assembly thereof in order to obtain the catalyst may be done by any means enabling the most complete amalgam possible. Thus, it may be performed by co-grinding or any other compounding technique. Also at this step, any other phase or one or several support material(s) may be added.

As indicated before, this method allows producing a mixed and multiphase oxides catalyst which has proven to be effective in many catalytic reactions, including: the oxidation of propene into acrolein, the oxidative dehydrogenation of butene into butadiene, the oxidation of isobutylene into methacrolein, the ammoxidation of propene into acrylonitrile and the ammoxidation of isobutylene into methacrylonitrile.

The disclosure also concerns a catalytic system comprising separately at least one active phase based on bismuth molybdate and one co-catalyst based on iron molybdate and at least one amongst cobalt and nickel. To be used, the separated phases are assembled as indicated before, for example by co-grinding.

This catalytic system which may be obtained according to the above-described method, comprises the following advantageous features, considered separately or in any combination:

the active phase meets the following stoichiometry BiₓMo_{y}O_{z} where 2>x/y>0.5 and z is comprised between 6 and 12; preferably this stoichiometry is selected amongst the following ones: Bi₂Mo3O_{12,} Bi₂Mo₂O9 and Bi₂MoO₆;

the active phase is activated by at least one alkali metal, preferably at least potassium; advantageously, only this phase is activated by an alkali metal;

the co-catalyst meets the following stoichiometry FeₓCo₁₋ₓMoO₄ where x is a decimal number such that 0<x<1, preferably such that 0.5≤x≤0.9;

the active phase content is lower than or equal to 50 weight % with respect to the weight of the catalytic system, preferably it varies from 10% to 45%.

The disclosure also concerns any use of a catalytic system as defined hereinabove, in particular for at least one amongst the following catalytic reactions: the oxidation of propene into acrolein, the oxidative dehydrogenation of butene into butadiene, the oxidation of isobutylene into methacrolein, the ammoxidation of propene into acrylonitrile and the ammoxidation of isobutylene into methacrylonitrile.

In order to illustrate the implementations of the different advantages of the disclosure, some terms/expressions are defined.

By mixture of the precursors of mixed oxides in a solvent, it should be understood in particular a solution or a suspension of said precursors in the solvent.

By isolation of the catalyst, or of any of its phases, in particular the active phase or the co-catalyst, it should be understood all treatment operations well known to those skilled in the art, such as retrieval from the liquid hydrothermal reaction medium, washing, drying, and any other treatment leading to the separation of the catalyst or of any of its phases, for the use thereof in a catalytic reaction.

In the following examples, the activity of catalysts of the disclosure and of a catalytic system of the disclosure is compared with that of a so-called industrial catalyst, that is to say prepared by calcination. The production method thereof was as follows:

Ammonium heptamolybdate (NH₄)₆Mo₇O₂₄ has been used as a molybdenum precursor, whereas all of the other metals other than cations have been added in the form of nitrates. Since the solubility of the precursors in water is very high, the desired concentration ranges have been easy to obtain. Besides, since bismuth nitrate is immediately hydrolyzed into bismuth oxynitrate which is not soluble in water, Bi₅O(OH)₉(NO₃)₄, and in order to promote the complete dissolution of bismuth with is barely soluble in water, a first solution has been prepared by dissolving 2g of tartaric acid in 100 ml of demineralized water that has been acidified beforehand with 1.5 ml of nitric acid (65%). After the addition of bismuth nitrate, the solution has been heated to 60° C. and kept under stirring for 30 min, until a colorless and clear solution is obtained. The addition of iron, cobalt and potassium in the form of nitrate has been executed in this order and each new salt has been added only after the complete dissolution of the previous one. Finally, the solutions 1 and 2 have been mixed under stirring and kept at 60° C. for 3 hours. The resulting suspension has been completely evaporated in a furnace at 120° C. Finally, the obtained product has been calcined at 350° C. for 2 hours to decompose the residual nitrates and then heated to 500° C. at the rate of 5° C/min and kept at this temperature for 2 hours.

### EXAMPLE 1

### Synthesis of a catalyst of the invention through a microwave-assisted hydrothermal reaction

The prepared catalyst meets the formula BiMoFeCoK, it has been produced as follows:

Bismuth acetate (or nitrate), iron nitrate and cobalt (or/and nickel) nitrate have been dissolved in 10 ml of H₂O so as to form the solution 1. Next, a stoichiometric amount of ammonium heptamolybdate has been dissolved in 10 ml of H₂O so as to form the solution 2. Afterwards, the solution 1 has been added slowly to the solution 2 so as to form a suspension, which has been kept under stirring for 1 hour. The pH of the mixture has been set to 1.8 by the supplemental HNO₃ or NH₄OH. Then, the suspension has been heated up to 150° C. by irradiation with microwaves and kept at this temperature for 10 minutes in a first step. Once the first step is completed, KOH is added and the mixture is basified to pH 8.5 by adding ammonia 32%. Then, the suspension has been heated up to 200° C. by irradiation with microwaves and kept at this temperature for 30 minutes. The obtained solid has been retrieved by centrifugation, washed twice with 10 ml of H₂O and once with 10 ml of ethanol and finally dried for 16 hours at 120° C.

The following variations have been tested: solvents, pH, reaction time (2 minutes to 96 h), irradiation temperature (150-240° C.), stoichiometry BiₓFe_{y}Co_{z}Ni_{c}Mo_{b}Kₐ with 0≤a, b, c, x, y, z≤15.

An implementation of the method is represented in the following Table 1:

TABLE 1

| | |
|---|---|
| Bismuth acetate (CH₃CO₂)₃Bi, | |
| ammonium heptamolybdate | |
| (NH₄)₆Mo₇O₂₄ | |
| cobalt nitrate Co(NO₃)₂•2H₂O | Iron nitrate Fe(NO₃)₃ |
| [symbol] | [symbol] |

| | |
|---|---|
| Step 1 | Stirring for 1 hour |
| | Setting of the pH with HNO₃ |
| | Heating with microwaves to 150° C., for 10 min |
| Step 2 | +KOH and pH setting with NH4OH |
| | Heating with microwaves to 200° C., for 30 min |
| | Centrifugation and washing |

### EXAMPLE 2

### Synthesis of a catalytic system of the invention through a microwave-assisted hydrothermal reaction

The industrial catalysts currently contain several typical phases MoO_{3,} Bi₂Mo₃O₁₂, Fe₂(MoO₄)₃, FeₓCo₁₋ₓMoO₄, NiMoO₄, Bi₂Mo₂O_{9 . . . .}

The two most useful phases are FeₓCo₁₋ₓMoO₄and Bi₂Mo₃O₁₂. Since the iron/cobalt/molybdenum mixed phase is very difficult to synthesize through the precipitation/calcination method because of the oxidation of Iron II into Iron III, the synthesis of this phase on an industrial scale cannot be considered. Indeed, Fe₂(MoO₄)₃ is always formed.

It has been possible to synthesize the two phases according to a method of the disclosure involving a microwave-assisted hydrothermal synthesis, which allows getting rid of the oxidation of iron, through the following protocol:

Bi₂Mo₃0₁₂

Bismuth nitrate and nitric acid have been dissolved in 150 ml of bi-distilled water. A second solution has been prepared by dissolving the ammonium heptamolybdate in 100 mL of bi-distilled water. Afterwards, the two solutions have been mixed and the resulting mixture has been kept under stirring for 10 minutes at 300 rpm, the pH has been set to 1 by addition of ammonium hydroxide, it has been transferred afterwards into a 1 L Teflon vial for irradiation with microwaves.

The microwave-assisted hydrothermal synthesis has been performed at 150° C. for 10 minutes. After treatment with microwaves, the collected product has been retrieved by centrifugation at 3000 rpm, and then washed twice with de-ionized water and ethanol. Finally, the sample has been dried at 90° C. for 8 hours.

FeₓCo₁₋ₓMoO₄ with 0.5<x<0.9

A first solution of sodium molybdate has been dissolved in 250 mL of bi-distilled H₂O. Afterwards, iron II chloride and cobalt nitrate hexahydrate have been dissolved in 250 ml of triethylene glycol. Afterwards, the two solutions have been mixed and the resulting solution has been kept under stirring for 10 minutes at 300 rpm. Then, the solution is transferred into a 1 L Teflon vial for irradiation with microwaves. The microwave-assisted hydrothermal synthesis has been carried out at 150° C. for 10 min. After treatment with microwaves, the collected product has been retrieved by centrifugation at 3000 rpm, then washed twice with water and ethanol. Finally, the sample has been dried at 90° C. for 8 hours.

### EXAMPLE 3

In this example, the catalyst prepared by microwaves has been tested in the controlled oxidation reaction of propene into acrolein and compared at iso-mass with the industrial catalyst prepared by calcination. The stoichiometry of the catalyst is: Mo₁₂Co_{7.12}Fe_{1.8}Bi_{0.65}Kₓ.

The test is done under a gas stream constituted by C₃H₆/O₂/N₂: 1/1.5/8.6, for a total gas flow rate of 60 ml/min with 250 mg of catalyst and at 350° C. Table 2 hereinbelow provides the propene conversion and the acrolein selectivity after 48h under a gas stream.

TABLE 2

| | Industrial catalyst | Microwaves catalyst |
|---|---|---|
| Propene | 11% | 34% |
| conversion | | |
| Acrolein | 94% | 92% |
| selectivity | | |

### EXAMPLE 4

In this example, catalysts prepared by microwaves and having different stoichiometries in Mo have been compared to one another in the controlled oxidation reaction of propene into acrolein.

The test is done under a gas stream constituted by C₃H₆/O₂/N₂: 1/1.5/8.6, for a total gas flow rate of 60 ml/min with 250 mg of catalyst and at 350° C. Table 3 hereinbelow provides the propene conversion and the acrolein selectivity after 48h under a gas stream.

TABLE 3

| | Mo₈Co_{7.12} | Mo₁₀Co_{7.12} | Mo₁₂Co_{7.12} |
|---|---|---|---|
| | Fe_{1.8}Bi_{0.65}Kₓ | Fe_{1.8}Bi_{0.65}Kₓ | Fe_{1.8}Bi_{0.65}Kₓ |
| Propene | 2% | 5% | 34% |
| conversion | | | |
| Acrolein | 69% | 95% | 92% |
| selectivity | | | |

### EXAMPLE 5

In this example, catalysts prepared by microwaves and having different stoichiometries in Bi have been compared to one another in the controlled oxidation reaction of propene into acrolein.

The test is done under a gas stream constituted by C₃H₆/O₂/N₂: 1/1.5/8.6, for a total gas flow rate of 60 ml/min with 250 mg of catalyst and at 350° C. Table 4 hereinbelow provides the propene conversion and the acrolein selectivity after 48h under a gas stream.

TABLE 4

| | Mo₁₂Co_{7.12} | Mo₁₂Co_{7.}12 | Mo₁₂Co_{7.12} |
|---|---|---|---|
| | Fe_{1.8}Bi_{0.65}Kₓ | Fe1_{.8}Bi_{0.5}Kₓ | Fe_{1.8}Bi_{0.2}Kₓ |
| Propene | 34% | 10% | 11% |
| conversion | | | |
| Acrolein | 92% | 93% | 94% |
| selectivity | | | |

### EXAMPLE 6

In this example, the catalyst prepared by microwaves has been tested in the controlled oxidation reaction of propene into acrolein. The stoichiometry of the catalyst MW is: Mo₁₂Co_{7.12}Fe_{1.8}Bi_{0.65}Kₓ.

The test is done under a gas stream constituted by C₃H₆/O₂/N₂: 1/1.5/8.6, for a total gas flow rate of 60 ml/min with 250 mg of catalyst and at 350° C. Table 5 hereinbelow provides the propene conversion and the acrolein selectivity after 358h under a gas stream.

TABLE 5

| | Microwaves catalyst |
|---|---|
| Propene conversion | 22% |
| Acrolein selectivity | 96% |

### EXAMPLE 7

In this example, the catalyst prepared by microwaves with nickel has been tested in the controlled oxidation reaction of propene into acrolein. The stoichiometry of this catalyst is: Mo₁₂Co₄Ni_{3.12}Fe_{1.8}Bi_{0.65}Kₓ.

The test is done under a gas stream constituted by C₃H₆/O₂/N₂: 1/1.5/8.6, for a total gas flow rate of 60 ml/min with 250 mg of catalyst and at 350° C. Table 6hereinbelow provides the propene conversion and the acrolein selectivity after 48h under a gas stream.

TABLE 6

| | Catalyst |
|---|---|
| | Mo₁₂Co₄Ni_{3.12}Fe_{1.8}Bi_{0.65}K_{x.} |
| Propene conversion | 11% |
| Acrolein selectivity | 92% |

### EXAMPLE 8

In this example, the catalyst prepared by microwaves is prepared by mechanical mixtures of the useful phases: FeₓCo₁₋ₓMoO₄ and Bi₂Mo₃0₁₂.

TABLE 7

| weight % | weight % | Propene | Acrolein |
|---|---|---|---|
| Fe_{0.67}Co_{0.33}MoO₄* | Bi₂Mo₃O₁₂ | conversion (%) | selectivity (%) |
| 100 | 0 | 2 ± 1% | 21 ± 2% |
| 90 | 10 | 68 ± 1% | 77 ± 2% |
| 80 | 20 | 70 ± 1% | 73 ± 2% |
| 70 | 30 | 73 ± 1% | 76 ± 2% |
| 60 | 40 | 70 ± 1% | 74 ± 2% |
| 0 | 100 | 6 ± 1% | 74 ± 2% |

*FeₓCo₁₋ₓMoO₄ with x = 0.67

The test is done under a gas stream constituted by C₃H₆/O₂/N₂: 1/1.5/8.6, for a total gas flow rate of 60 ml/min with 250 mg of catalyst and at 350° C. Table 7 hereinbelow provides the propene conversion and the acrolein selectivity after 48h under a gas stream.

1. A method for producing a multiphase mixed-oxide catalyst comprising at least one active phase based on bismuth molybdate and one co-catalyst based on iron molybdate and at least one amongst the two elements cobalt and nickel, said method including the following steps:
preparing a mixture of the precursors of said mixed oxides in a solvent,
making said precursors react through a microwave-assisted hydrothermal reaction, and
isolating the mixed oxides to obtain the catalyst.

2. The production method according to claim 1, wherein said precursors are reacted through a microwave-assisted hydrothermal reaction in two steps, a first microwave-assisted hydrothermal reaction and a second microwave-assisted hydrothermal reaction, between which the pH of the reaction mixture obtained from the first microwave-assisted hydrothermal reaction is set to 8-8.5.

3. The production method according to claim 1, wherein the catalyst comprises molybdenum oxide.

4. The production method according to claim 1, wherein the catalyst is supported, said method comprising the addition of one or several support material(s), before the microwave-assisted hydrothermal reaction(s).

5. The production method according to claim 1, wherein the microwave-assisted hydrothermal reaction(s) is/are performed at a temperature from 150° C. to 240° C.

6. The production method according to claim 1, wherein the microwave-assisted hydrothermal reaction(s) is/are carried out over a time period from 2 minutes to 10 hours.

7. The production method according to claim 1, wherein the active phase of the catalyst meets the following stoichiometry BiₓMo_{y}O_{z} where 2>x/y>0.5 and z is comprised between 6 and 12;
wherein this stoichiometry is selected amongst Bi₂Mo₃O₁₂, Bi₂Mo₂O₉, and Bi₂MoO₆.

8. The production method according to claim 1, wherein the active phase of the catalyst is activated by at least one alkali metal.

9. The production method according to claims 1, wherein the co-catalyst meets the following stoichiometry FeₓCo₁₋ₓMoO₄ where x is a decimal number such that 0<x<1.

10. The production method according to claim 1, wherein the catalyst meets the formula MoₘCoₙNiₚFe_{q}BiᵣMₛ where M is an alkali metal, m, n, p, q, r and s are natural or decimal numbers, with m varying from 1 to 5, n and p varying independently of one another from 0 to 1 with n+p different from 0, 0<q≤1, 0<r≤3 and 0<q≤0.2.

11. The production method according to claim 1, wherein:
a mixture of the precursors of the active phase in a solvent, on the one hand, and a mixture of the precursors of the co-catalyst in the same solvent or in another solvent, on the other hand, are prepared;
the precursors of the active phase and the precursors of the co-catalyst are reacted separately, through a microwave-assisted hydrothermal reaction;
the active phase and the co-catalyst, respectively, are isolated; and
the active phase and the co-catalyst are assembled to obtain the catalyst.

12. The production method according to claim 1, wherein the mixture(s) of the precursors of the mixed oxides is/are obtained in one or more of the solvents selected from water, organic solvents and any combination of said organic solvents together or with water.

13. The production method according to claim 11, wherein the catalyst is supported, said method comprising the addition of one or several support material(s), and/or the catalyst comprises molybdenum oxide, said method comprising the addition of molybdenum oxide, the addition of said support material(s) and/or of the molybdenum oxide being carried out during the synthesis or the assembly of the active phase and of the co-catalyst to obtain the catalyst.

14. The production method according to claim 1, for the production of a multiphase mixed-oxide catalyst intended for at least one amongst the following catalytic reactions: the oxidation of propene into acrolein, the oxidative dehydrogenation of butene into butadiene, the oxidation of isobutylene into methacrolein, the ammoxidation of propene into acrylonitrile and the ammoxidation of isobutylene into methacrylonitrile.

15. A catalytic system comprising separately at least one active phase based on bismuth molybdate and one co-catalyst based on iron molybdate and at least one amongst cobalt and nickel.

16. The catalytic system according to claim 15, wherein the active phase meets the following stoichiometry BiₓMo_{y}O_{z} where 2>x/y>0.5 and z is comprised between 6 and 12; wherein this stoichiometry is selected amongst Bi₂Mo₃O₁₂, Bi₂Mo₂O₉, and Bi₂MoO₆.

17. The catalytic system according to claim 15, wherein the active phase is activated by at least one alkali metal.

18. The catalytic system according to claim 15, wherein the co-catalyst meets the following stoichiometry FeₓCo₁₋ₓMoO₄ where x is a decimal number such that 0<x<1.

19. The catalytic system according to claim 15, wherein the active phase content is lower than or equal to 50 weight % with respect to the weight of the catalytic system.

20. A use of a catalytic system according to claim 15, for at least one amongst the following catalytic reactions: the oxidation of propene into acrolein, the oxidative dehydrogenation of butene into butadiene, the oxidation of isobutylene into methacrolein, the ammoxidation of propene into acrylonitrile and the ammoxidation of isobutylene into methacrylonitrile.
